(19) United States
(12) Patent Application Publication
(10) Pub. No.: US 2008/0038374 A1
(43) Pub. Date: Feb. 14, 2008

(54) New Use

(75) Inventors: Mona Stahle, Stockholm (SE); Gunther Weber, Solna (SE); Johan Heilborn, Stockholm (SE)

Correspondence Address: BIRCH STEWART KOLASCH & BIRCH, PO BOX 747, FALLS CHURCH, VA, 22040-0747, US

(73) Assignee: Lipopeptide AB, Stockholm (SE)

(21) Appl. No.: 11/632,553

(22) PCT Filed: Jul. 26, 2005
(86) PCT No.: PCT/SE05/01184

§ 371 Date: May 11, 2007

(30) Foreign Application Priority Data
Jul. 28, 2004 (SE) 0401943-6

(51) Int. Cl. A61K 31/047; A61K 33/06; A61N 5/06; A61P 17/00; A61P 31/00; A61P 9/00; C12N 5/02

(52) U.S. Cl. 424/682; 435/383; 514/729; 607/88

(57) ABSTRACT

The present invention relates to the use of a vitamin D compound active in up-regulating the expression and production of hCAP18 in humans for the manufacture of a medicament for treatment of conditions deficient in or benefiting from LL-37. The vitamin D active compound can be used as a medicament for treatment of injured tissues, chronic ulcers, burn wounds, skin infections and atopic dermatitis and for improving the microvasculature. UVB irradiation can also be used to up-regulate hCAP18.

### FIELD OF THE INVENTION

The present invention relates to the use of a vitamin D compound, which is able to specifically and directly up-regulate hCAP18, for the manufacturing of a medicament with antimicrobial effect for treatment of conditions deficient in LL-37, such as chronical ulcers, and atopic dermatitis.

### BACKGROUND OF THE INVENTION

Epithelia constitute the primary barrier between host and the potentially harmful environment, and therefore the protection of this interface is vital. A wound represents a broken barrier and immediately sets in motion a series of tightly orchestrated events with the purpose to promptly reinstate the integrity of the barrier. Urgent wound closure has evolved in higher organisms, diverging from the time-consuming process of complete regeneration of tissue seen in lower species. Impaired wound healing represents a major challenge in clinical medicine ranging from the relative delay in “normal” healing seen with increasing age to pathologic non-healing ulcers.

Antimicrobial peptides are effector molecules of the innate immune system, which serve to protect the host against potentially harmful microorganisms. They are conserved through evolution and are widespread in nature. In human, only a handful has been identified so far; among which the defensins and the human cathelicidin antimicrobial peptide hCAP18 have been implicated in epithelial defence (Selsted et al., J Biol Chem 258:14485-14489, 1983).

WO 96/08508 relates to the human polypeptide FALL-39, as well as to pharmaceutical compositions containing said peptide and having an antimicrobial activity against bacteria. The peptide was named FALL-39 after the first four amino acid residues and consisted of the 39 amino acid C-terminal part of a proprotein concomitantly identified by three separate groups (Cowland et al., FEBS, 1995; Agerberth et al., Proc Natl Acad Sci USA 1995; Larrick et al., FEBS Letters 1996). The peptide was shown to have potent anti-microbial activity against both gram-positive and gram-negative bacteria. Further characterization of the C-terminal peptide demonstrated a shorter sequence comprising 37 amino acids excluding the first two (FA) resulting in LL-37, which is the accepted current designation (Gudmundsson et al., Eur J Biochem 238:325-332, 1996).

The proprotein was named hCAP18, human cationic anti-microbial protein, and is a member of the cathelicidin family of proteins consisting of cathelin, which has been conserved through evolution and a C-terminal part, variable in different species. In man, hCAP18 is the only member of this protein family, whereas in other species, such as mouse and pig, there are several members. LL-37 is an endogenous peptide, which is released by proteolytic cleavage of hCAP18; the C-terminus is LL-37. LL-37 is thought to function extracellularly and there is no evidence for intra-cellular cleavage of the pro-protein. hCAP18/LL-37 is present in leukocytes and in barrier organs such as skin, mucous membranes, respiratory epithelium and reproductive organs. The localization of hCAP18/LL-37 to barrier epithelia seems to be consistent with a protective role for the peptide in preventing local infection and systemic microbial invasion. In association with inflammation hCAP18/LL-37 is upregulated in skin epithelium (Frohm et al., J Biol Chem 272:15258-15263, 1997) and mucous membranes (Frohm Nilsson et al., Infect Immun 67:2561-2566, 1999).

Heilborn et al., J Invest Dermatol 120:379-389, 2003 (Frohm Nilsson Thesis 2001) concomitantly demonstrated that human cathelicidin anti-microbial protein, hCAP18, is induced in skin wounding, with high levels and release of active C-terminal peptide, LL-37, in physiological healing but not in chronic non-healing ulcers. HCAP18/LL-37 was induced in the wound edge epithelium during normal wound healing, but was not detected in the ulcer edge epithelium of chronic leg ulcers, only in the wound bed and stromal tissue. It was suggested that low levels of hCAP18 and lack of active LL-37 in chronic ulcers contribute to impaired healing. It has also been shown that hCAP18 is induced during re-epithelialization of organ-cultured skin wounds, and that this re-epithelialization was inhibited by antibodies against LL-37 in a concentration-dependant manner. These findings suggest that LL-37 plays a crucial role in wound closure. Although a therapeutic use of LL-37 has been suggested, this has so far not been realized.

Chronic ulcers in contrast to acute wounds constitute a major clinical problem and although our understanding of the physiologic wound process has increased over the past decades only minor therapeutic improvements have been attained. Distinct etiologies may underlie the development of ulcerations in different clinical conditions but, what-ever the cause, non-healing ulcers are characterized by an inability of the epithelium to migrate, proliferate and close the barrier defect. The most common type of chronic skin ulcers is leg ulcers due to venous insufficiency. These patients develop peripheral venous oedema with subsequent ulceration of the skin, whereas the arterial circulation is intact. Leg and foot ulcers due to arteriosclerotic deficiencies and metabolic disorders, such as diabetes, are less common.

In addition, skin ulcers develop in association with immune diseases such as pyoderma gangrenosum and vasculitis. Current treatment includes long-term systemic immunosuppression and is not always effective. Epithelial defects and ulcers in the oral, genital and gastrointestinal mucous membranes are common and cause much distress. The underlying pathomechanisms are not always clear, such as in aphtae and erosive lichen, and treatment is poor.

The terms dermatitis and eczema, respectively, which terms are used interchangeably, encompass a variety of distinct conditions with different etiological background, such as allergic and non-allergic contact dermatitis, nummular eczema, sebborhoic eczema and atopic eczema.

Atopic eczema or in other words atopic dermatitis is a chronic, itchy inflammatory skin disease affecting approximately 20% of children in Western societies. The etiology is unknown but a combination of genetic and environmental factors are considered to interplay to manifest the disease. Atopic eczema lesions are characterized by a defect in skin barrier and the patients are prone to skin infections. The skin of patients with atopic eczema is frequently colonized with bacteria such as Staphylococcus aureus and the patients often require treatments with antibiotics. Effective topical antibacterial treatment is lacking. This is in contrast to psoriasis, another inflammatory skin disease, where the skin seems to be rather protected against infections. Further, it has been reported (Ong et al, New Engl. J. Med., 347(15), 1151 (2002)) that innate antimicrobial peptides including LL-37 are upregulated in psoriasis but suppressed in atopic eczema and that patients with atopic dermatitis showed a deficiency in the expression of LL-37.

Impaired angiogenesis and deficient blood supply is important in many diseases, for instance in chronic ulcers, and burn wounds. Koczulla, et al., J. Clin. Invest. 111:1665-1672 (2003) concludes that LL-37/hCAP18 induces functionally important angiogenesis.

Vitamin D refers to a number of vital fat-soluble steroid hormones, such as cholecalciferol (vitamin D₃) and ergocalciferol (vitamin D₂). Cholecalciferol is obtained from animal food, and ergocalciferol is produced in plants and yeast. Said two forms of vitamin D are metabolised in the same way, first hydroxylated into 25-OH-D, which compound is then 1-hydroxylated into 1,25-(OH)₂-D, the biologically most active metabolite. The chemical formulas of vitamin D₂ and vitamin D₃ are given in FIG. 1.

Vitamin D has for long been known for its important role in regulating body levels of calcium and phosphorus, and in mineralization of bone. More recently, it has become clear that receptors for vitamin D are present in a wide variety of cells, and that this hormone has biologic effects which extend far beyond control of mineral metabolism.

As a transcriptional regulator of bone matrix proteins, vitamin D induces the expression of osteocalcin and suppresses synthesis of type I collagen. In cell cultures, vitamin D stimulates differentiation of osteoclasts. However, studies of humans and animals with vitamin D deficiency in mutations in the vitamin D receptor suggest that these effects are perhaps not of major physiologic importance, and that the crucial effect of vitamin D on bone is to provide the proper balance of calcium and phosphorus to support mineralization.

The classical manifestations of vitamin D deficiency are rickets, which is seen in children and results in bony deformities including bowed long bones. Deficiency in adults leads to the disease osteomalacia. Both rickets and osteomalacia reflect impaired mineralization of newly synthesized bone matrix, and usually result from a combination of inadequate exposure to sunlight and decreased dietary intake of vitamin D.

Vitamin D₃ has also been reported to be involved in insulin secretion (C. Cade et al, Endocrinology, 120, 1490 (1987), prolactin synthesis (J. D. Wark et al, J. Biol. Chem., 258, 270 (1983), epidermal cell differentiation (J. Hasami et al, Endocrinology, 113, 1950 (1983) and in cancer (K. Chida et al, Cancer Res., 45, 5426 (1985).

### PRIOR ART

K. V. Ramesh et al., Indian J. Exp. Biol., 31, 778 (1993) reported in a short communication that cholecalciferol increases wound breaking strength and accelerates re-epithelization of acute wounds in Wistar rats after intraperitonial administration of cholecalciferol. Detailed experimental conditions are not given.

In DE 10161729 is disclosed an ointment for use in wound healing and for treatment of eczema, containing as active ingredients zinc oxide, cod-liver oil comprising vitamin A and vitamin D₃, woolfat and paraffin.

In U.S. Pat. No. 4,610,978 are disclosed compositions containing 1-alpha-hydroxycholecalciferol or 1α,25-dihydroxy-cholecalciferol for the topical treatment of skin disorders such as dermatitis and psoriasis. The used dosages were between 0.03-1.0 μg/g of composition to avoid side effects. The results in Table I show that no effect on dermatitis could be obtained with ergocalciferol, cholecalciferol or 24,25-dihydroxycholecalciferol, but a marked effect with 1α-OH-cholecalciferol and 1α,25-di(OH)₂-cholecalciferol. The type of dermatitis is not stated.

WO 9105537 discloses methods for enhancing wound healing including gastric ulcer healing by administration of high doses of a vitamin D compound. Both topical and other administration forms are claimed. A large number of compounds such as vitamin D₂, vitamin D₃, 5,6-epoxy derivatives of vitamin D and fluoro derivatives are listed but data are only available for 1,25-dihydroxy-cholecalciferol on puncture, that is acute, wounds in rats using dosages up to 54 μg/g oil.

Wang et al., Journal of Immunology, 2004, 173:2909-2912, discloses that 1,25-(OH)₂D₃ directly induces the expression of hCAP18 in keratinocytes and leukocytes in vitro.

### SUMMARY OF THE INVENTION

The present invention is based on the finding that 25-hydroxy vitamin D₃ (25-OH-D₃), and 1,25-dihydroxy vitamin D₃ (1,25-(OH)₂-D₃) surprisingly, specifically and directly, stimulate the upregulation of hCAP18 and the biosynthesis of the antimicrobial peptide LL-37.

### BRIEF DESCRIPTION OF THE DRAWINGS

FIG. 1 shows the chemical formulas of vitamin D₂ and D₃, respectively.

FIG. 2 is a staple diagram showing the expression of hCAP18 RNA in human primary keratinocytes after treatment with vitamin D₃ and analogues.

FIG. 3 shows a Western blot analysis of protein extracts from keratinocytes treated with 1 μM 1,25-(OH)₂-D₃.

FIG. 4 is a staple diagram showing the concentration dependence of hCAP18 stimulation by vitamin D₃.

FIG. 5 is a staple diagram showing that there is no significant effect of 7-dehydrocholesterol on hCAP18 expression.

FIG. 6 is a staple diagram showing that the expression of hCAP18/LL37 is up-regulated by vitamin D₃ in human skin in vivo.

FIG. 7 is a staple diagram showing that hCAP18 mRNA is up-regulated by vitamin D in acute wounds.

FIG. 8 shows a Western blot analysis of hCAP18 and LL-37 protein extracts from acute wounds treated with calcipotriol

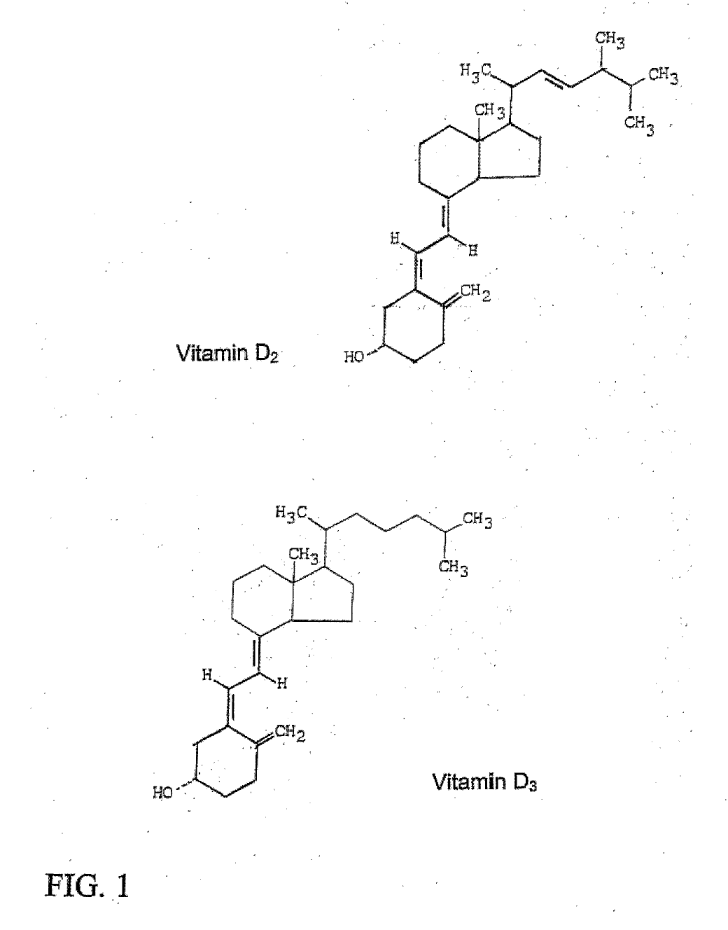

### DESCRIPTION OF THE INVENTION

The present invention refers to the use of a vitamin D compound active in up-regulating the expression and production of hCAP18 in humans for the manufacture of a medicament for treatment of conditions deficient in or benefiting from LL-37.

Vitamin D compounds, which can be used in accordance with the invention, are vitamin D compounds which up-regulate the expression and production of hCAP18 in the assays as described in Example 1. Examples of said compounds can be selected from the group consisting of cholecalciferol (D₃), 25-hydroxy-cholecalciferol (25-OH-D₃), 1,25-dihydroxy-cholecalciferol (1,25-(OH)₂-D₃), 1,25-dihydroxyergocalciferol (1,25-(OH)₂-D₂), as well as other vitamin D active metabolites, and vitamin D active synthetic analogues.

Examples of vitamin D active metabolites are, in addition to 25-OH-D₃ and 1,25-(OH)₂-D₃, 24,25-(OH)₂-D₃ and 1,24,25-(OH)₃-D₃ and also 25-OH-D₂ and 1,25-(OH)₂-D₂, 24,25-(OH)₂-D₂ and 1,24,25-(OH)₃-D₂.

Vitamin D active synthetic vitamin D analogues are for instance calcipotriol, calcitriol, tacalcitol, maxacalcitol and others, for instance as described in WO 02/34235.

The active vitamin D compounds also activate the VDRs, vitamin D receptors. Vitamin D has a direct effect by binding the VDRE, vitamin D responsive element, in the hCAP18 promotor. We have shown that the crucial VDRE is located at −494/−480 in the promotor (Weber et al., J. Invest Dermatol 124(5): 1080-2).

A preferred vitamin D compound is 25-hydroxy-chole-calciferol (25-(OH)-D₃), or 1,25-dihydroxy-cholecalciferol (1,25-(OH)₂-D₃).

The use of a vitamin D compound to enhance the endogenous production of LL-37 is a safe way of providing the antimicrobial peptide LL-37 to a site in need of said peptide.

The up-regulation of hCAP18 by vitamin D compound in skin epithelial cells is potentiated by the preaddition to the cells of a calcium salt. The invention therefore also refers to the use of a vitamin D₃ compound in combination with a calcium salt for treatment of conditions deficient in LL-37.

The invention refers to the use of a vitamin D active compound in a sufficient amount for stimulating the endogenous production of the antimicrobial peptide LL-37 in human cells, especially epithelial cells. The up-regulation of hCAP18 by the vitamin D compound and the biosynthesis of the antimicrobial peptide LL-37 is obtained using a relatively low concentration, such as 10 nM-1 μM of a vitamin D compound.

The vitamin D compound is preferably locally administered. In systemic administration there is always a risk of hypercalcemia. When locally administered the vitamin D compound is preferably applied to the skin or membrane in an amount of 0.05-10 μg/cm², preferably in an amount of 0.1-0.5 μg/cm².

The invention especially refers to the use of a vitamin D compound for the manufacture of a medicament having an antimicrobial effect.

The invention also refers to the use of a vitamin D compound for the manufacture of a medicament providing a sustained and enhanced antimicrobial protection in injured tissue.

The invention also refers to the use of a vitamin D compound for the manufacture of a medicament for the prophylactic and curative treatment of infections in connection with atopic dermatitis.

The invention also refers to the use of a vitamin D compound for the manufacture of a medicament for healing of wounds, especially chronic ulcers, such as ulcers due to venous insufficiency, ulcers due to arteriosclerotic deficiency, ulcers due to diabetes, and burns.

The invention also refers to the use of a vitamin D compound for the manufacture of a medicament for improving microvasculature through stimulation of angiogenesis.

A pharmaceutical composition comprising a vitamin D compound, as mentioned above, in combination with a pharmaceutically acceptable carrier can be used to facilitate administration of the compound, systemically or locally.

Suitable routes for administration may include topical, rectal, transdermal, vaginal, intestinal, transmucosal and oral administration, parenteral delivery, including intramuscular, subcutaneous, and intracutaneous injections.

Pharmaceutically acceptable carriers enable the formulation of tablets, pills, capsules, powders, liquids, gels, syrups, slurries, suspensions or creams, ointments, solutions, patches or any other suitable type of formulation.

In a pharmaceutical composition for topical administration the amount of the vitamin D compound can be 1-100 μg/g of the composition, and preferably 5-50 μg/g.

A pharmaceutical composition can be formulated with carriers comprising in addition to the active substance a liquid or solid filler, diluent, excipient, solvent or encapsulating material, involved in carrying or transporting the vitamin D compound from the application area to the cells or site of treatment. The carrier must be compatible with the other ingredients of the composition and not injurious to the patient.

Some examples of materials which can be used in a pharmaceutically acceptable carrier are: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; cocoa butter and suppository waxes; oils, such as peanut oil, cottonseed oil, safflower oil, sesame oil, olive oil, corn oil and soybean oil; membrane lipids, such as phospho-lipids and galactolipids; glycols, such as propylene glycol; polyols, such as glycerine, sorbitol, mannitol and poly-ethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; wetting agents, emulsifiers and lubricants, such as sodium lauryl sulfate and magnesium stearate, as well as coloring agents, release agents, coating agents, sweetening, flavoring and perfuming agents, preservatives and antioxidants; and other non-toxic compatible substances.

By means of the present invention it will be possible to increase the rate of wound healing in a human subject by administering a therapeutically effective amount of a vitamin D compound, to stimulate the endogetous production of the antimicrobial peptide LL-37. In the same way it will be possible to treat an angiogenetic disorder, and an atopic eczema.

The invention also refers to the use of UVB irradiation for treatment of conditions deficient in or benefiting from LL-37, such as acute and chronic wounds, burn wounds, skin infections and atopic dermatitis. It has recently been demonstrated that irradiation with a single dose of UVB, 280-320 nm, but not of UVA, 340-400 nm, significantly up-regulated the expression of hCAP18 and vitamin D receptor (DR) in the skin of healthy volunteers.

The invention also refers to a method of enhancing the expansion of human autologous cells in vitro, wherein cells are isolated from an excised-piece of healthy tissue, said isolated cells are cultivated in vitro in a growth medium, and the cultivated cells are subsequently harvested and used for tissue repair, which is characterised in that a vitamin D compound active in up-regulating the expression and production of hCAP18 in humans is added to the growth medium.

### EXAMPLES

The invention is exemplified, but not limited in scope, by the following examples.

### Example 1

### Vitamin D Up-Regulates the Expression of hCAP18 mRNA and Protein in Human Keratinocytes

The following experiments show details for the proven up-regulation of hCAP18, and can be used as a test for demonstrating the ability of a vitamin D compound to up-regulate hCAP18.

Human neonatal epidermal keratinocytes were obtained from Cascade Biologics, Inc. (Lot # 1C1145; Port-land, OR) and cultured in EpiLife serum-free keratinocyte growth medium (Cascade Biologics, Inc.) containing growth supplements and a calcium concentration of 0.06 mM. Cells at passage 3 were allowed to grow for three days to approximately 60% confluence. For calcium switch experiments, 1.5 mM CaCl₂ (added from a 1 M stock solution) was added to the plates and after 48 hours the cells were regarded as differentiated (observed as an induction of mRNA expression of the two keratinocyte markers transglutaminase and cellular retinoic acid-binding protein II). The compounds assayed were 25-hydroxy-cholecalciferol (25-OH-D₃), 1,25-dihydroxycholecalciferol (1,25-(OH)₂-D₃), and calcipotriol/MC 903 (Leo Pharmaceutical, Denmark). Their effect on hCAP18 expression was monitored by quantitivate PCR analysis.

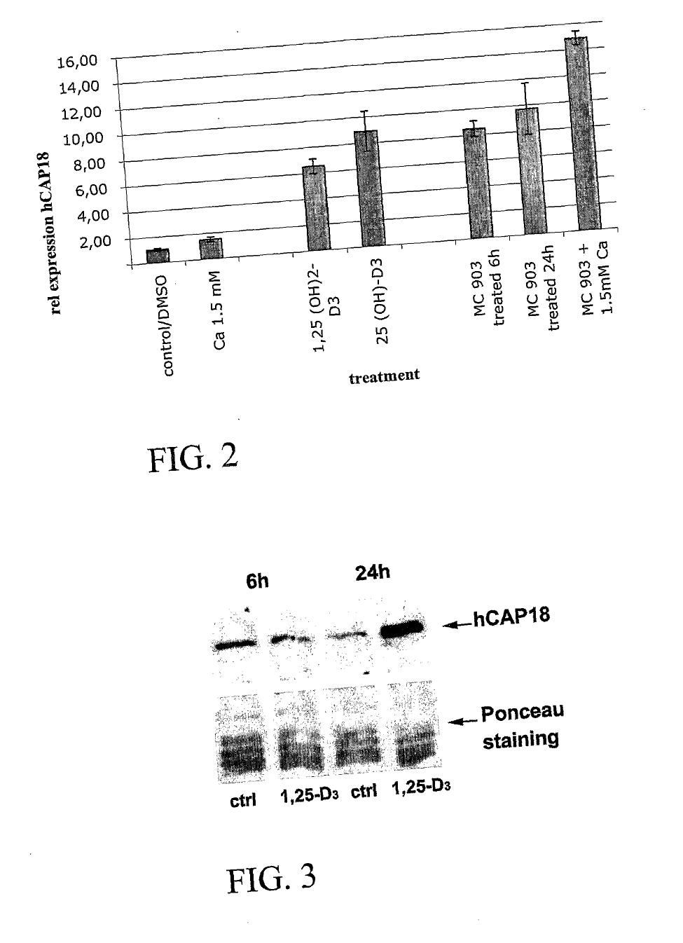

FIG. 2 shows the expression of hCAP18 mRNA in human primary keratinocytes after treatment with 1,25-dihydroxy-D₃, 25-hydroxy-D₃ and MC 903. Keratinocytes were grown as above and treated with said compounds for 4 h. RNA was extracted with the RNeasy kit (Qiagen) and reverse transcribed with a first strand synthesis kit (Amersham-Pharmacia). RNA was quantified by Real-Time PCR on an ABI Prism 7700 (Applied Biosystems) using 5 ng of cDNA according to standard protocols. The samples were evaluated in triplicates. Sequences were 5′-GTCACCAGAGGATTGTGACTTCAA-3′ and 5′-TTGAGGGTCACTGTCCCCATA-3′ for the primers, and 5′-CCGCTTCACCAGCCCGTCCTT-3′ for the fluorigenic probe. The samples were normalized by quantification of 18S-RNA (Assay on Demand, Applied Biosystems).

The induction of differentiation by calcium increased the expression by about 1.5 fold. The vitamin D compounds assayed here, up-regulated hCAP18 by about one magnitude. The transcription of hCAP18 was already up-regulated after 6 h treatment, indicating an event of early regulation.

Protein extracts were assayed by Western blot analysis to verify that the elevated level of hCAP18 mRNA was reflected on the protein level. Consistent with a delay in time course from an induction of hCAP18 transcription to protein synthesis, increased levels of hCAP18 protein were not observed after 6 hours but evident after 24 hours treatment.

FIG. 3 shows a Western blot analysis of protein extracts from keratinocytes treated with 1 μM 1,25-(OH)₂-D₃. Cells were grown and treated with 1,25-(OH)₂-D₃ as described above, and extracted in SDS-containing sample buffer according to standard protocols. For the detection of hCAP18 protein, the extracts were separated on a 15% Tris-Glycine gel. To confirm that equal amounts of protein in each sample were blotted, the filters were reversibly stained with a 3% Ponceau S (Sigma) solution in 3,% TCA, before incubating with the primary antibody. Affinity purified anti-LL37 antiserum (Heilborn et al., supra) was used at a 1:1000 dilution. After electroblotting onto nitrocellulose filters (Schleicher & Schuell), and sequential incubation with primary antibodies and horseradish-peroxidase conjugated IgG (SantaCruz Biotechnology), signals from enhanced chemiluminiscence (Amersham) were captured with a CCD camera (LAS 1000, Fujifilm).

Taken together, these data lead to the hypothesis that all tested vitamin D compounds directly up-regulated hCAP 18 expression on the transcriptional level.

### Example 2

### Vitamin D Compounds Stimulate the Expression of hCAP18 at Physiological Concentrations

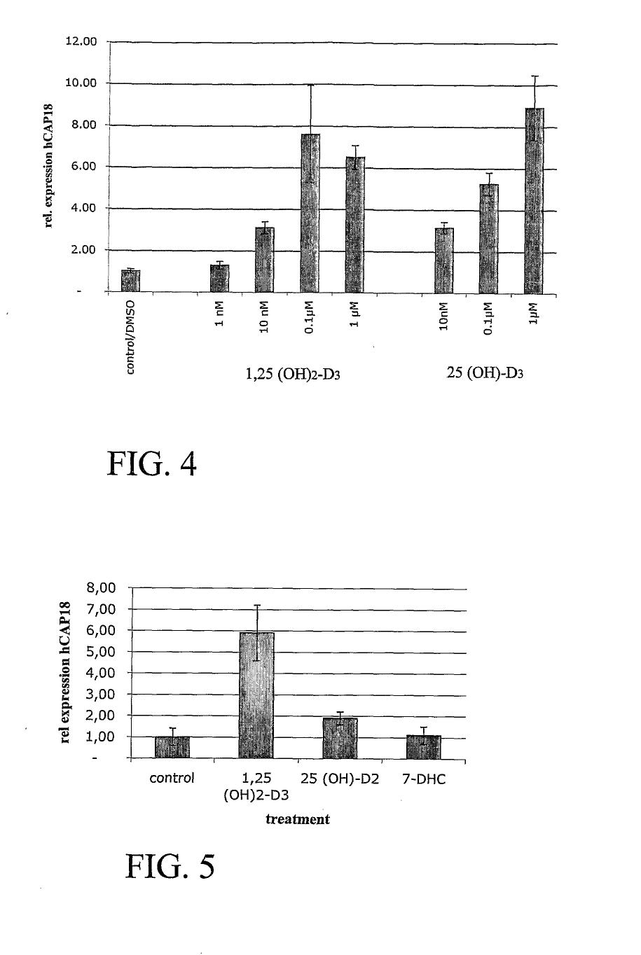

To assess whether physiological levels were sufficient to induce the stimulation of the hCAP18 promoter, the study was performed at the different concentrations 1, 10, 100 and 1000 nM of 1,25-(OH)₂-D₃ and 25-OH-D₃ (FIG. 4). The highest activity was observed at 0.1 and 1.0 μM, however a substantial portion of stimulation remained down to the level of 10 nM. The physiologic serum concentration of 25-OH-vitamin D₃ is between 20 nM and 125 nM, whereas the 1,25-(OH)₂-D₃ is present in pM concentration. A dosage form of a vitamin D compound for topical administration includes for instance about 1-100, preferably 5-50 μg vitamin D compound per gram composition.

### Example 3

### Specificity of Vitamin D Compounds

To assess the specificity of the vitamin D regulation on the hCAP18 gene, the vitamin D₃ precursor 7-dehydrocholesterol (7-DHC) was assayed at a concentration of 1 μM as in Example 1. 7-DHC had no effect on hCAP18 expression. FIG. 5 shows that there is no significant effect of 7-dehydrocholesterol on hCAP18 expression. Assays were performed and measured as above. The effect of 1,25-(OH)₂-D₃ is shown for comparison. 1,25-(OH)₂-D₂ was also tested and was found to have an activity comparable to 1,25-(OH)-D₃.

### Example 4

### 1,25-(OH)₂-D₃ Up-Regulates the Expression of hCAP18 and the Production of the Mature Antimicrobial Peptide LL-37 in Human Skin In Vivo

To assay whether 1,25-(OH)₂-D₃ stimulated hCAP18 expression in vivo, we applied 1,25-(OH)₂D₃ to the skin of three healthy individuals.

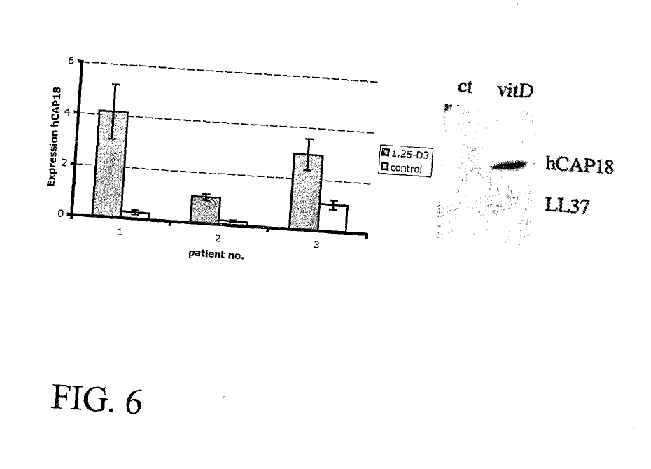

A stock solution of 4 mM 1,25-dihydroxyvitamin D₃ in isopropanol was diluted in ethanol:propylene glycol [70:30] to a final vitamin D concentration of 0.002%. The vehicle containing ethanol 96% and propylene glycol [70:30] with 0.5 mg/ml BHT (butylated hydroxy toluene) served as control. In a room with dim light, the vitamin D and the control were locally applied to the skin (the right and left upper arm respectively) of three healthy volunteers. The test area of 2×2.5 cm on each arm was cleaned with saline solution (NaCl 0.9) before application. Evaporation time for the solutions of vitamin D and the vehicle varied between 8 and 15 minutes. The tested area was then covered with plastic film and band-aid (Gladpack, Melolin and Mefix). After 4 days the band-aid and the plastic film was removed and the test area was rapidly cleaned with 40% ethanol. Punch biopsies (4 mm) were obtained from the test area (after infiltration anesthesia with 2-3 ml Xylocaln with epinefrin) and were frozen instantly. FIG. 6 shows that the expression of hCAP18/ll-37 is up-regulated by vitamin D₃ in human skin in vivo. Left panel: Real-Time PCR expression analysis on cDNA from skin biopsies of three probands. The biopsies were cut in 50 μm sections, placed in RNA Later solution and extracted for RNA as described above. Right panel: Western blot analysis on proband no. 3. Frozen biopsies from one healthy volunteer were cut in 50 μm sections and extracted for protein. Cold extraction buffer of 60% aqueous acetonitrile containing 1% trifluoroacetic acid (Frohm et al, 1996) was added and samples were eluted on an Eppendorff shaker (IKA-Vibrax-VXR, Labasco, Mölndal, Sweden) and then centrifuged. The supernatants were lyophilized and then diluted in 1000 μl of double distilled H₂O. Protein concentrations were measured by Protein Assay Kit (Bio-Rad Laboratories, Hercules Calif.) based on the Bradford method (Bradford, 1976) and samples were diluted to a final protein concentration of 1 mg per ml. The Western blot analysis was performed as described above.

### Example 5

### Vitamin D Enhances the Up-Regulation of the Protein hCAP18/LL-37 During Wounding in Human Skin In Vivo

To assess whether treatment with a vitamin D compound can further enhance the up-regulation of hCAP18 during acute skin injury, fresh surgical wounds in healthy volunteers (n=9) were treated with vitamin D ointment.

The investigation was non-randomized, single blinded including four females and five males, age 22-30 years. Only fair skinned, young and healthy individuals were included and the tissue was obtained from the same body location in a non-sun exposed area. Each individual obtained 3 wounds in the left and the right inguinal region, respectively. The topical treatment with vitamin D was applied on one side and the control treatment on the contra lateral side. To each of the wounds on one side, 25 μg calcipotriol in 0.5 g ointment (Daivonex, LEO Pharma, Malmo, Sweden) was applied to a total test area of 2×2.25 cm, including one wound with surrounding intact skin. The control wounds in the opposite inguinal region were treated with vaseline (ACO, Stockholm, Sweden). All wounds were covered with inert dressing (Melolin, Smith and Nephew, Hull, U K; Mefix, Molnlycke A B, Gothenburg, Sweden; Tegaderm, 3M Health Care, St. Paul, USA). After 12 hours the dressing was changed and the treatment was repeated. In the first four individuals investigated the bandage was removed at 24 hours and the wounds were excised with a 6 mm biopsy punch and snap frozen. In the following five individuals biopsies were obtained from intact skin at 0 hours in addition to the wounds excised at 24 hours. These five individuals were treated with vitamin D and control for totally 24 hours as described above. The bandage was then removed, the test area was cleaned with saline solution and the remaining wounds were covered with inert dressing and subsequently excised 48 hours post-wounding.

Expression of hCAP18 mRNA was quantified by Real-Time PCR, as described in Example 1.

Protein was calculated from frozen biopsies from patients (n=5) cut in 50 μm sections and homogenized. Protein was extracted in SDS-containing sample buffer according to standard protocols (Ausubel et al, 2003) alternatively by a buffer of 60% acetonitrile containing 1% trifluoroacetic acid (Heilborn et al, 2003). Protein concentrations were measured by Protein Assay Kit (Bio-Rad Laboratories, Hercules, USA) based on the Bradford method (Bradford 1976).

For the detection of hCAP18 protein, the extracts were separated on a 18% Tris-Tricine gel (Schagger and von Jagow 1987). The total protein amount in each sample was corrected to 5 μg. Affinity purified anti-LL-37 antiserum (Heilborn et al, 2003) was used at a 1:1000 dilution. After electroblotting onto PVDF membranes (BioRad, Hercules, Calif.), and sequential incubation with primary antibodies and horse-radish-peroxidase conjugated IgG (SantaCruz Biotechnology, Santa Cruz, Calif.), signals from enhanced chemiluminiscence (Amersham Biosciences, Piscataway, N.J.) were captured with a CCD camera (LAS 1000, Fujifilm). To confirm that equal amounts of protein in each sample that were blotted, the membranes were stained with 0.1% Amidoblack 10B (Sigma) solution in methanol/acetic acid/H₂O at 45/10/45 (v/v).

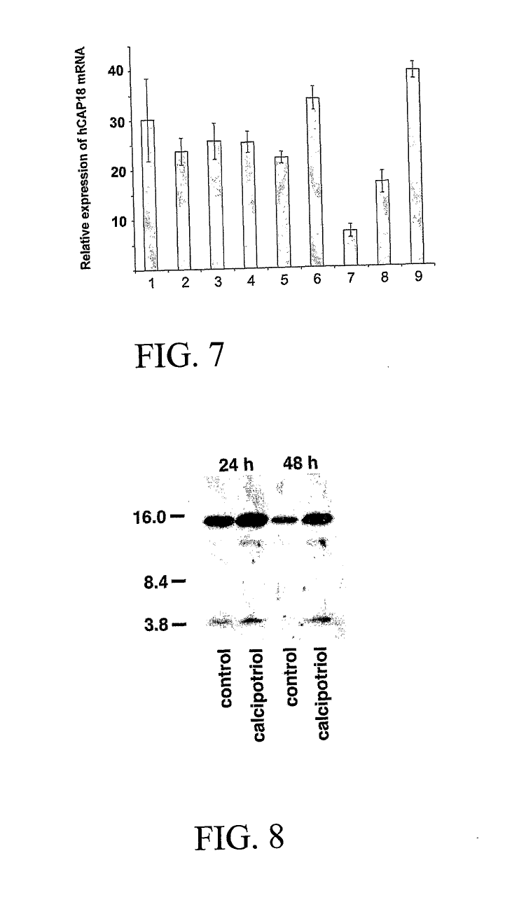

FIG. 7 shows Real-Time RT-PCR expression analysis of the nine probands (no. 1-9) at 24 hours, showing that topical vitamin D treatment enhances the up-regulation of hCAP18 mRNA in acute wounds. RNA was extracted from excision biopsies of acute wounds locally treated with calcipotriol or vaseline (control) for 24 h. The stimulation of hCAP18 gene expression after treatment is shown in arbitrary units and standardized to 18S RNA expression. For each individual, values are presented relative to the expression of hCAP18 mRNA of the respective control wound, which is set as 1 (not shown).

FIG. 8 shows that vitamin D treatment enhances the up-regulation of hCAP18 and the processed peptide LL-37 in acute wounds. By immunoblotting, three of the five individuals investigated demonstrated stronger immunoreactive bands, corresponding to the intact non-processed 18 kDa holoprotein, for the calcipotriol treated wounds, compared with the bands of the control wounds. Overall the strongest bands for hCAP18 were detected at 24 hours, but by 48 hours the difference between the wounds treated with calcipotriol and the control wounds was even more pronounced. In addition, in all three individuals, stronger immunoreactive bands, corresponding to the processed peptide LL-37, were present in wounds treated with calcipotriol compared to the control wounds. In normal intact skin, hCAP18 protein was barely detectable, with a week band in the holoprotein region but no band for LL-37 (data not shown).

Thus it can be concluded that treatment with vitamin D ointment significantly increases the level of hCAP18/LL-37 protein in acute wounds thereby providing a sustained antimicrobial activity.

### Example 6

### Up-Regulation of hCAP 18 in Chronic Ulcers

Tissues

Patients (n=9) with chronic (>6 months duration) leg ulcers due to venous insufficiency were recruited at the Department of Dermatology, Karolinska Hospital, Stockholm. Individuals with a history of diabetes mellitus, arterial insufficiency or chronic inflammatory disease were excluded. Patients with signs of eczema in the ulcer margin, clinical signs of infection or undergoing systemic or topical anti-biotic treatment at the time for biopsy were also excluded. Patients included were all treated with inert local dressings (Melolin, Smith and Nephew, Hull, U K) and standard compression bandaging prior to the topical vitamin D treatment at 0 and 12 h. Calcipotriol (25 μg) in 0.5 g ointment (Daivonex, LEO Pharma, Malmo, Sweden) was applied to a test area of 2×2.25 cm localized in the wound margin of the chronic ulcers, including 50% of the epithelialized area. Vaseline (ACO, Stockholm, Sweden) served as control. Punch-biopsies (4 mm) were obtained at 24 h from the wound margin (including 50% of the epithelialized area) and frozen instantly. All participants gave their written informed consent. The study was approved by the Regional Committee of Ethics and was conducted according to the Declaration of Helsinki Principles.

Expression Analysis

Frozen biopsies from patients (n=9) were cut in 50 μm sections. RNA was extracted with the Qiagen RNeasy kit (Operon Biotechnologies, Cologne, Germany) and reverse transcribed with a first strand synthesis kit (Amersham Biosciences, Norwalk, Conn.). RNA was quantified by Real-Time PCR as described in Example 1.

The results are expressed as arbitrary units comparing the expression of hCAP18 mRNA in untreated and treated biopsies from the same patient. The results are presented as average of triplicates and standard deviations in the following Table 1.

TABLE 1
Up-regulation of hCAP18 mRNA after treatment with Daivonex

| | |
|---|---|
| Average | Standard deviation |

| Patient | Control | Daivonex | Control | Daivonex |
|---|---|---|---|---|
| V13 | 13.7 | 69.7 | 1.8 | 7.0 |
| V14B | 16.8 | 49.9 | 1.5 | 4.1 |
| V15 | 2.8 | 39.7 | 0.3 | 6.8 |
| V16 | 7.0 | 26.2 | 1.1 | 2.5 |
| V17 | 1.7 | 80.9 | 0.2 | 8.8 |
| V18 | 5.3 | 16.0 | 0.8 | 1.7 |
| V19 | 2.2 | 23.8 | 0.1 | 1.6 |
| V20 | 9.4 | 77.2 | 4.4 | 6.7 |
| V21 | 10.3 | 128.3 | 1.7 | 18.4 |

Studies are planned for evaluating the long-term effect of this treatment on the improvement of healing.

Tests have shown that there is a lack of LL-37 in the epithelial cells also in diabetic ulcers, and additional tests are to be performed with additional types of chronic ulcers.

### Example 7

### Up-Regulation of hCAP 18 in Atopic Dermatitis

To assay whether 1,25-(OH)₂-D₃ stimulated hCAP18 expression in atopic dermatitis, we applied 1,25-(OH)₂-D₃ on the skin of four individuals, two females and two men, with active atopic dermatitis. The patients had suffered from the eczema for 11-30 years. The diagnosis was made by a dermatologist. A stock solution of 4 mM 1,25-(OH)₂-D₃ in isopropanol was diluted in ethanol:propylene glycol (70:30) to a final concentration of 0.002%. To prevent deactivation of 1,25-(OH)₂-D₃, the procedure was performed in a room with dim light. The 1,25-(OH)₂-D₃ were locally applied on lesional and non-lesional skin area of patients. The skin surface was cleaned with saline solution (NaCl 0.9) before application. The treated skin area was measured to 2×2.25 cm. Evaporation time for the solutions of 1,25-(OH)₂-D₃ varied between 8 and 15 minutes. The skin area was then covered with plastic film and band-aid (Quickpack: Haushalt und Hygiene GmbH, Renningen, Germany; Melolin: Smith and Nephew, Hull, UK; Mefix: Molnlycke A B, Gothenburg, Sweden). After 4 days the band-aid and the plastic film were removed and the skin areas were rapidly cleaned with 40% ethanol. Punch biopsies (4 mm) were obtained from the treated skin areas (after infiltration anesthesia with 2-3 ml Xylocaln with epinephrin) and frozen instantly. On the first day, control biopsies were also obtained from non-treated lesional and non-lesional skin and the tissues were snap-frozen as described. RNA was extracted and hCAP18 mRNA expression determined by Real-Time PCR as described above in Example 1. The results are presented in Table 2 below.

TABLE 2
Up-regulation of hCAP18 mRNA after
treatment with 1,25-(OH)₂-D₃

| | Non-lesional skin | | | Lesional skin | | |
|---|---|---|---|---|---|---|
| Patient | Un- | | Increase^{a} | Un- | | Increase^{a} |
| No. | treated | Treated | % | treated | Treated | % |
| 1 | 2.87 | 24.2 | 841 | 6.5 | 64 | 977 |
| 2 | 1.76 | 8.1 | 459 | 13.2 | 29 | 220 |
| 3 | 5.64 | 739.4 | 13104 | 1.8 | 1093 | 5638 |
| 4 | 4.7 | 24.8 | 520 | 4.4 | 539 | 79756 |

^{a}Increase = treated/untreated

Tests are planned for investigation of the effect of vitamin D treatment on the microflora, especially on Staphylococcus aureus, in atopic eczema patients.

1. Use of a vitamin D compound active in up-regulating the expression and production of hCAP18 in humans for the manufacture of a medicament having an antimicrobial effect.

2. Use according to claim 1 for the manufacture of a medicament providing a sustained and enhanced antimicrobial protection in injured tissue.

3. Use according to claim 1 for the manufacture of a medicament for treatment of infections in atopic dermatitis.

4. Use of a vitamin D compound active in up-regulating the expression and production of hCAP18 in humans for the manufacture of a medicament for improving microvasculature through stimulation of angiogenesis.

5. Use according to claim 1, wherein said vitamin D compound is selected from the group consisting of cholecalciferol (D₃), 25-hydroxy-cholecalciferol (25-(OH)-D₃), 1,25-dihydroxy-cholecalciferol (1,25-(OH)₂-D₃), 1,25-dihydroxy-ergocalciferol (1,25-(OH)₂-D₂), other vitamin D active metabolites, and vitamin D active synthetic analogues.

6. Use according to claim 5, wherein the vitamin D active metabolite is selected from the group consisting of 24,25-dihydroxy-cholecalciferol (24,25-(OH)₂-D₃), 1,24,25-trihydroxy-cholecalciferol (1,24,25-(OH)₃-D₃), 25-hydroxy-ergocalciferol (25-(OH)-D₂), 24,25-dihydroxy-ergocalciferol, (24,25-(OH)₂-D₂) and 1,24,25-trihydroxy-ergocalciferol (1,24,25-(OH)₃-D₂).

7. Use according to claim 5, wherein the vitamin D active synthetic analogue is selected from the group consisting of calcipotriol, calcitriol, tacalcitol and maxacalcitol.

8. Use according to claim 7, wherein the vitamin D active synthetic analogue is calcipotriol.

9. Use according to claim 1, wherein the vitamin D compound is used in an amount sufficient to stimulate the endogenous production of the antimicrobial peptide LL-37 in human cells.

10. Use according to claim 1, wherein the vitamin D compound is used in an amount sufficient to stimulate the endogenous production of the antimicrobial peptide LL-37 in epithelial cells.

11. Use according to claim 9, wherein the cells are treated with a calcium salt before the addition of the vitamin D compound.

12. Use according to claim 1, wherein the medicament is locally administered.

13. Use according to claim 12, wherein the vitamin D compound is used in an amount of 0.05-10 μg/cm² skin area, preferably in an amount of 0.1-0.5 μg/cm² skin area.

14. Use of UVB irradiation for treatment of conditions deficient in or benefiting from LL-37, such as acute and chronic wounds, burn wounds, skin infections and atopic dermatitis.

15. Method of enhancing the expansion of human autologous cells in vitro, wherein the cells are isolated from an excised piece of healthy tissue, said isolated cells are cultivated in vitro in a growth medium, and the cultivated cells are subsequently harvested and used for tissue repair, characterised in that a vitamin D compound active in up-regulating the expression and production of hCAP18 in humans is added to the growth medium.
